(19) United States
(12) Patent Application Publication
(10) Pub. No.: US 2005/0155408 A1
(43) Pub. Date: Jul. 21, 2005

(54) Sensor arrangement

(76) Inventors: Helmut Weyl, Schwieberdingen (DE); Hans-Martin Wiedenmann, Stuttgart (DE); Juergen Wilde, Fellbach (DE); Johannes Kanters, Yokohama (JP)

Correspondence Address: KENYON & KENYON, ONE BROADWAY, NEW YORK, NY, 10004, US

(21) Appl. No.: 10/506,642

(22) PCT Filed: Feb. 17, 2003
(86) PCT No.: PCT/DE03/00463

(30) Foreign Application Priority Data
Mar. 8, 2002 (DE) 102 10 313.5

(51) Int. Cl. G01N 37/00; G01N 33/00; G01M 13/00

(52) U.S. Cl. 73/23.31; 73/866.5

(57) ABSTRACT

A sensor system having a sensor for measuring a gas parameter of a test gas via a measuring element which is accommodated in a housing and projects therefrom at least on the test gas side at a protruding section, and a test-gas line, through which the test gas flows, having a sensor insertion opening and a receiving element for the housing surrounding the insertion opening and attached to the test-gas line. For the purpose of assembly-independent, reproducible alignment of the measuring element with respect to the test-gas flow during assembly of the sensor system, the receiving element bears an internal thread, the piercing point of which is oriented with respect to the test-gas flow, and the housing bears an external thread, which is able to be screwed into the internal thread and the piercing point of which is oriented with respect to the measuring element. The housing is fixed via a predefined tightening torque in the receiving element.

### FIELD OF THE INVENTION

The present invention relates to a sensor system.

### BACKGROUND INFORMATION

In conventional sensor configurations, e.g., lambda probes for measuring oxygen concentration in exhaust gas from internal combustion engines, exact measured-value acquisition requires the protruding end of the measuring element or sensor that is immersed in the test gas and is located on the test gas side to be aligned in a predefined manner relative to the gas flow. This alignment is performed on-site during assembly.

In the case of a conventional sensor system of this type, as described in German Patent Application No. DE 43 18 107, such an alignment of a lambda probe relative to the exhaust-gas flow is performed during assembly so that a gas inflow opening formed in a protective pipe covering the protruding end of the measuring element on the test gas side is located on the side away from the exhaust-gas flow. As a result, condensation water included in the exhaust gas is not able to reach the protruding end of the measuring element, is not able to deposit there, and is consequently not able to affect the measuring accuracy of the lambda probe. To always ensure correct alignment of the lambda probe during installation in the exhaust-gas pipe in a reproducible manner regardless of the qualifications of the on-site assembler, an assembly aid is provided such that a marking indicating the position of the gas inflow opening on the protective pipe is made on a metal sleeve that is fixedly connected to the housing and covers a connection-side protruding end of the measuring element that protrudes from the housing. This marking allows the sensor to be inserted into the receiving element on the exhaust-gas pipe such that the gas inflow opening in the protective pipe points in the exhaust-gas flow direction. After the lambda probe achieves an orientation coordinated with the exhaust-gas flow direction, the housing is tightened via a union nut in a receiving element that is attached to the exhaust-gas pipe and that accommodates the housing. It is proposed as an alternative assembly aid to provide the sensor housing with such a geometry that assembly is only possible with the desired alignment of the lambda probe via a form-locking arrangement.

### SUMMARY

An example sensor system of the present invention may have the advantage that the performed fixing of the piercing points starting points of the threads in the two parts to be screwed together, namely the housing and the receiving element, and the stipulated tightening torque makes it possible to always produce the correct position of the measuring element regardless of assembly. Since the housing itself is screwed into the receiving element, additional elements, e.g., a union nut, for fixing the sensor in the receiving element that are considered to be losable parts are rendered unnecessary.

According to an advantageous embodiment of the present invention, a marking is situated on the housing that specifies an orientation for the installation of the measuring element and is oriented with respect to the piercing point of the outside thread on the housing. Since the housing is completed with external housing during manufacture of the sensor, the marking oriented with respect to the piercing point of the threads, e.g., a simple radial blind hole in the housing, ensures prior to insertion of the measuring element that the measuring element is inserted into the housing with the correct alignment.

An example embodiment of a sensor system according to the present invention may have the advantage that the necessary alignment of the sensor in the receiving element is able to be ensured for an existing sensor design having hollow screw fixing of the sensor in the receiving element by simply minimally changing the manufacturing method and without changing the sensor itself. In particular, when the projection is achieved according to advantageous embodiments of the present invention by an insertion ring pressed tightly against the housing or an insertion pin inserted radially into the housing, a structural change is only required for the sensor receiving element with respect to the axial groove to be cut on the front side.

### BRIEF DESCRIPTION OF THE DRAWINGS

The present invention is explained in further detail in the exemplary embodiments shown in the figures and in the following description.

FIG. 1 shows a side view of a sensor system, partially cut.

FIG. 2 shows a top view of an insertion ring in the sensor system of FIG. 1.

FIG. 3 shows a longitudinal section of the sensor system in FIG. 1.

FIG. 4 shows an enlarged representation of the section IV in FIG. 3.

FIG. 5 shows an enlarged representation of the section V in FIG. 3.

FIG. 6 shows the same representation as in FIG. 5 with a modification to the sensor system.

FIG. 7 shows a side view of a sensor system according to a further exemplary embodiment, partially cut.

### DETAILED DESCRIPTION OF EXAMPLE EMBODIMENTS

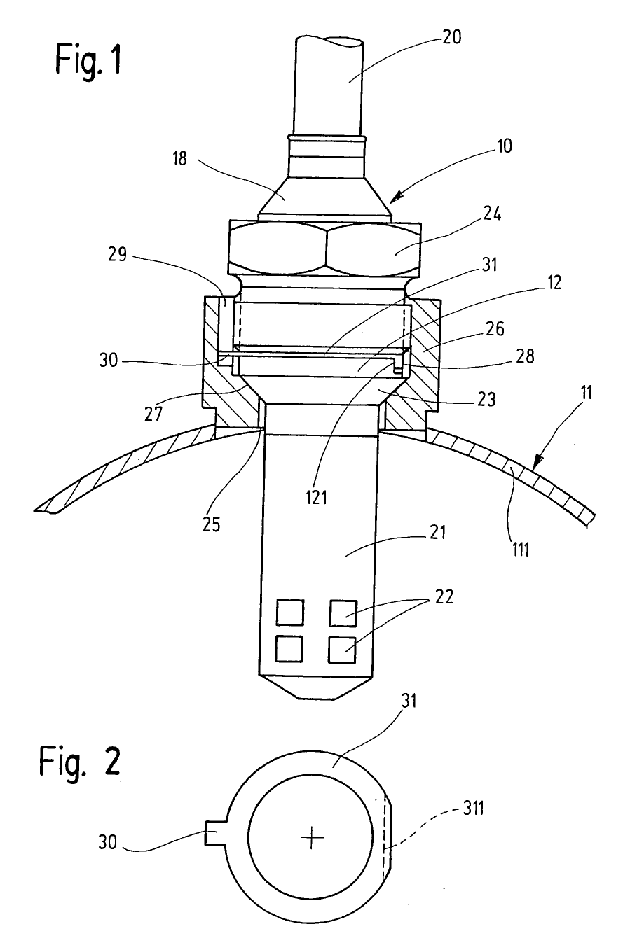

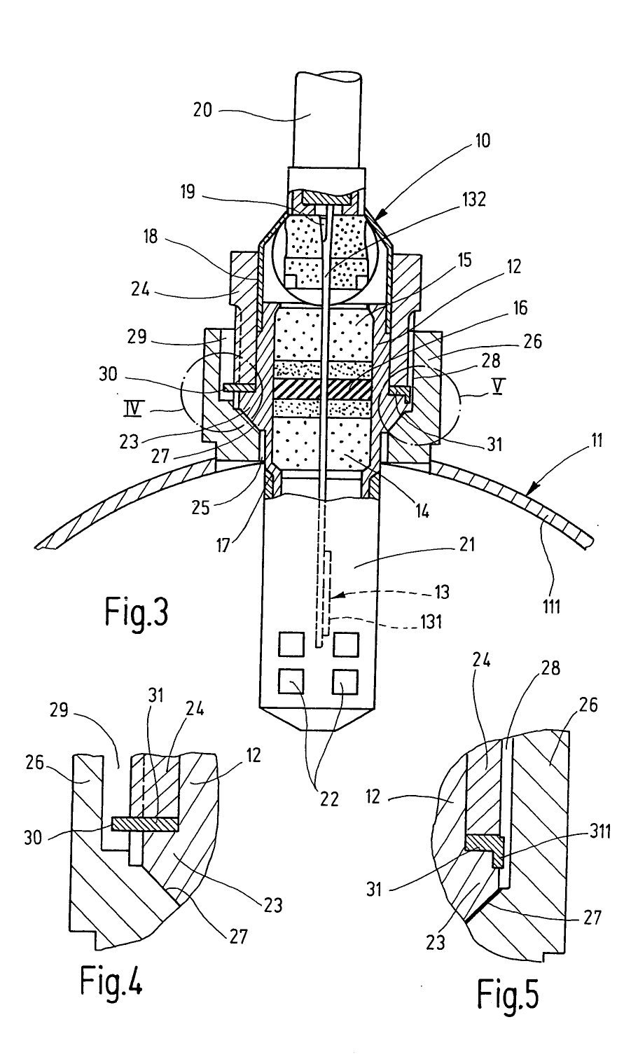

The sensor system shown in FIGS. 1 and 3 has a sensor 10 for measuring a gas parameter of a test gas, shown as a side view in FIG. 1 and as a longitudinal section in FIG. 3, and a test-gas line 11, through which a test gas flows, shown sectionally as a cross section in FIGS. 1 and 3. In the example in FIGS. 1 and 3, sensor 10 is a lambda probe for measuring oxygen concentration as the gas parameter in the exhaust gas of an internal combustion engine, and test-gas line 11 is an exhaust-gas pipe outgoing from one or more combustion cylinders of the internal combustion engine.

Sensor 10 has a sensor or a measuring element 13 (FIG. 3), which is accommodated in a housing 12 and protrudes therefrom at a section 131 on the test gas side and a section 132 on the connection side. In this context, measuring element 13 is surrounded by an electrically insulating ceramic insert 14 on the test gas side, an electrically insulating ceramic insert 15 on the connection side, and an interposed, packet-like seal 16, which is supported by the inner wall of housing 12. Ceramic insert 14 on the test gas side lies on a shoulder 17 on the inner housing wall, and the top housing edge is flared to ceramic insert 15 on the connection side. In an alternative embodiment, ceramic insert 15 on the connection side lies on a shoulder formed in housing 12, and the bottom housing edge is flared to ceramic insert 14 on the test gas side. As shown, the packet-like seal may be made up of three stacked sealing elements, the two external ones being made of steatite, for example, and the middle one being made of boron nitride. A metal sleeve 18, which covers an electrical clamp connector 19, which contacts connection-side section 132 of measuring element 13 and a connecting wire 20, is placed on housing 12. A protective pipe 21, which covers section 131 of measuring element 13 on the test gas side, is pushed onto the end of housing 12 facing away from metal sleeve 18. There are gas inflow openings 22 in protective pipe 21 so that after installation of sensor 10 in test-gas line 11, the test gas flowing in test-gas line 11 is able to flow through gas inflow openings 22 to measuring element 13. To mount sensor 10 on test-gas line 11, housing 12 is provided with a radial flange 23, which is beveled on the underside, and a hollow screw 24, which acts together with radial flange 23, is provided.

Test-gas line 11 has a sensor insertion opening 25, which is incorporated into line wall 111, and a receiving element 26 for housing 12 of sensor 10, which surrounds sensor insertion opening 25 and is attached to line wall 111. Receiving element 26 has a beveled support shoulder 27 for radial flange 23 of housing 12 and an internal thread 28, which corresponds with the external thread of hollow screw 24. As shown in FIGS. 1 and 3, receiving element 26 may be configured as a hollow connecting piece that is inserted into an expanded sensor insertion opening 25 in line wall 111 and is welded circumferentially to line wall 111. However, the hollow connecting piece may also be positioned on line wall 111 to surround sensor insertion opening 25 and welded with line wall 111.

During assembly of the sensor system, sensor 10 including its housing 12 is inserted into receiving element 26 at test-gas line 11, protective pipe 21 penetrating sensor insertion opening 25 into the interior of test-gas line 11, and housing 12 pushing so far into receiving element 26 that the beveled underside of radial flange 23 contacts beveled support shoulder 27 in receiving element 26. Hollow screw 24 is then pushed over metal sleeve 28 and housing 12 and screwed into internal thread 28 of receiving element 26 until its annular end face tightens radial flange 23 to support shoulder 27.

The accurate measuring function of sensor 10 requires section 131 of measuring element 13 on the test gas side projecting into the gas flow and surrounding protective pipe 21 to achieve a certain alignment within the test-gas flow. An assembly aid having an allocation element situated on housing 12 and oriented with respect to the installation position of measuring element 13 and an allocation element situated at receiving element 26 and oriented with respect to the test-gas flow is provided to ensure that the alignment is reproducible. In the exemplary embodiment in FIGS. 1-5, the allocation element disposed at receiving element 26 is an axial groove 29, which runs in the region of internal thread 28 and runs out freely on the front side, and the allocation element formed at housing 12 is a projection 30, which projects radially over the housing periphery, slides in a positive-locking manner in axial groove 29 when housing 12 is inserted into receiving element 26, and prevents rotation of sensor 10 about its axis. As FIG. 2 shows in particular, projection 30 is a one-piece part of an insertion ring 31, which lies on the flange surface of radial flange 23 facing hollow screw 24 and is fixedly connected to housing 12, preferably pressed against housing 12. Insertion ring 31 is pressed against housing 12 in that projection 30 is oriented with respect to the predetermined installation position of measuring element 13 in housing 12. In addition, housing 12 may have a flat section 121 at its periphery, and insertion ring 31 may be bent in its ring region 311, which is assigned to flat section 121, such that ring region 311 lies flat against flat section 121 (FIGS. 2 and 5). Bent ring region 311 is preferably positioned at insertion ring 31 such that it is diametrically opposed to projection 30 (FIGS. 1-3). When installing measuring element 13 in housing 12, flat section 121 is used to align measuring element 13 with respect to housing 12 in that measuring element 13 is oriented such that it has a certain rotation position with respect to flat section 121 of housing 12. If insertion ring 31 is then pushed onto housing 12, projection 30 has the desired orientation with respect to the alignment of measuring element 13 in housing 12.

Although not shown in greater detail, projection 30 may also be formed on housing 12 as one piece.

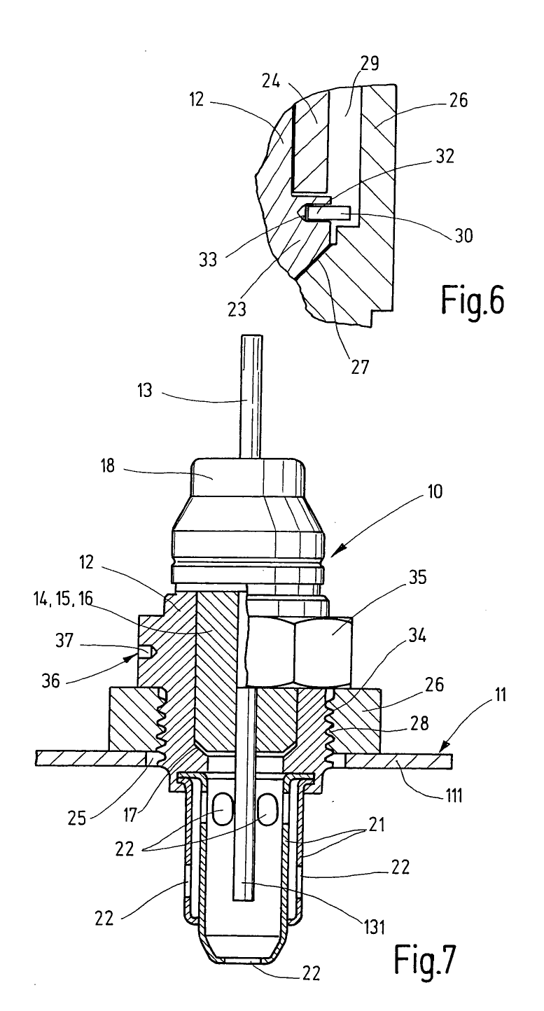

In the modification of the sensor system shown in FIG. 6, projection 30 is formed by an insertion pin 32, which is pressed into a radially formed blind hole 33 in housing 12. The part of insertion pin 32 protruding from blind hole 33 forms projection 30, which projects in a positive-locking manner into axial groove 29 in the groove width as described above.

In the sensor system shown in FIG. 7, the design of sensor 10 is modified to the extent that the hollow screw for fixing housing 12 in receiving element 26 is dispensed with, and housing 12 has its own external thread 34 as well as a hexagon 35 for screwing housing 12 into internal thread 28 of receiving element 26. To achieve the allocation elements at housing 12 and receiving element 26 for the purpose of exact alignment of measuring element 13 in test-gas line 11, the piercing point of external thread 34 at housing 12 is oriented with respect to the installation position of measuring element 13 in housing 12 and the piercing point of internal thread 28 in receiving element 26 is oriented with respect to the test-gas flow. This configuration of the piercing points of threads 28 and 34 allows housing 12 to always be screwed from only one specific rotation position about its longitudinal axis into receiving element 26. If a predefined tightening torque is additionally applied to hexagon 35 following complete screwing in of housing 12, measuring element 13 is aligned in the desired position in test-gas line 11. The tightening torque may be controlled via a torque wrench.

During the production process, housing 12 is first completed with external thread 34 and hexagon 35, and measuring element 13 including ceramic inserts 14, 15 and seal 16 are subsequently inserted into housing 12. To insert measuring element 13 into housing 12 during installation such that it has a certain orientation with respect to the piercing point of external thread 34, a marking 36, which is oriented with respect to the piercing point of external thread 34, is made on housing 12. Marking 36 is designed as a small radial blind hole 37, which is made in hexagon 35, in the exemplary embodiment in FIG. 7. Marking 36 is used to be able to fix measuring element 13 in housing 12 during the production process in a particularly simple manner such that the necessary orientation with respect to the piercing point of external thread 34 is achieved.

The present invention is not limited to the described sensor system having a lambda probe immersed in the exhaust-gas pipe of an internal combustion engine. Other sensors, e.g., temperature, moisture, or pressure sensors recording a corresponding parameter of the test gas, i.e., temperature, moisture, or pressure, may be used in the same manner instead of a lambda probe.

1-11. (canceled)

12. A sensor system, comprising:
a sensor to measure a gas parameter of a test gas;
a measuring element that is accommodated in a housing and projects therefrom at least on a test gas side at a protruding section;
a test-gas line, through which the test gas flows, the test gas line including a sensor insertion opening;
a receiving element for the housing which surrounds the insertion opening and is attached to the test-gas line; and
an assembly aid that reproducibly creates a predefined alignment of the protruding section of the measuring element in the test gas, and includes an allocation element situated at the housing and oriented with respect to an installation position of the measuring element, and further includes an allocation element situated at the receiving element and oriented with respect to the test-gas;
wherein the receiving element has an internal thread having a piercing point, the piercing point of the internal thread forming the allocation element oriented with respect to the test-gas,
wherein the housing has an external thread having a piercing point, which is able to be screwed into the internal thread, the piercing point of the external thread forming the allocation element oriented with respect to the measuring element, and
wherein the housing is fixed in the receiving element via a predefined tightening torque.

13. The sensor system as recited in claim 12, wherein the sensor is configured to measure an oxygen concentration in exhaust gas of an internal combustion engine, and the test-gas line is an exhaust gas pipe.

14. The sensor system as recited in claim 12, wherein a marking, which provides an orientation for the installation of the measuring element and is oriented with respect to the piercing point of the external thread, is positioned on the housing.

15. The sensor system as recited in claim 14, wherein the marking is a blind hole radially introduced into the housing.

16. The sensor system as recited in claim 12, wherein the receiving element has a housing support shoulder, and the housing has a radial flange that rests on the housing support shoulder and is configured to be tightened to the housing support shoulder via a hollow screw that overlaps the housing, and wherein the allocation element situated at the receiving element is an axial groove which runs in a region of the internal thread and runs out freely at a front end, and the allocation element situated at the housing is a projection which protrudes radially over a periphery of the housing and projects into the axial groove in a form-locking manner at least in a width of the groove.

17. The sensor system as recited in claim 16, wherein the projection is integrally formed in one piece on the housing.

18. The sensor system as recited in claim 16, wherein the projection is part of an insertion pin fixed in a radial bore hole in the housing.

19. The sensor system as recited in claim 16, wherein the projection is part of a flat insertion ring, which lies on the housing on a flange surface of the radial flange facing the hollow screw and is configured to be connected to the housing in a rotationally fixed manner.

20. The sensor system as recited in claim 19, wherein the insertion ring is pressed against the housing.

21. The sensor system as recited in claim 19, wherein the periphery of the housing has a flat section, and the insertion ring is bent in a ring region allocated to the flat section such that the ring region lies flat against the flat section.

22. The sensor system as recited in claim 21, wherein the bent ring region lies diametrically opposite the projection.

23. The sensor system as recited in claim 12, wherein the receiving element is a hollow connecting piece that is insertable into a wall opening into a line wall of the test-gas line and is welded to the line wall.
